(19) United States
(12) Patent Application Publication
(10) Pub. No.: US 2017/0079735 A1
(43) Pub. Date: Mar. 23, 2017

(54) SURGICAL INSTRUMENT INTENDED TO RECEIVE A CUTTING TOOL IN A REMOVABLE MANNER, SURGICAL KIT AND METHOD FOR MANUFACTURING THE SAME

(71) Applicant: IN2BONES, Ecully (FR)

(72) Inventors: Christian Baertich, Toulouse (FR); Joël Vernois, Picquigny (FR); Adrien Ray, Carouge (CH); David Jonathan Redfern, Hove (GB)

(73) Assignee: IN2BONES, Ecully (FR)

(21) Appl. No.: 15/267,861

(22) Filed: Sep. 16, 2016

(30) Foreign Application Priority Data
Sep. 17, 2015 (FR) FR-15 58777

(51) Int. Cl. A61B 50/30; A61B 17/02; A61B 17/16; A61B 17/32

(52) U.S. Cl. CPC A61B 50/3001; A61B 17/32; A61B 17/0218; A61B 17/1659

(57) ABSTRACT

Surgical instrument intended to receive a cutting tool in a removable manner, surgical kit and method for manufacturing the same are described. A surgical instrument (1) intended to receive a cutting tool in a removable manner includes a receiving member (7), including a body (8) provided with a means (10) for receiving the tool, which is designed to evolve between a locked configuration and an unlocked configuration, and a locking/unlocking device (14) comprising a first element (15) linked to the receiving member (7) by a helical connection. The locking/unlocking device (14) comprises a second element (22) linked to the receiving member (7) by a sliding connection, so that a relative rotation of the first (15) and second (22) elements results in a relative translation of the receiving member (7) and the second element (22) thereby allowing controlling the receiving means (10).

The present invention concerns the field of surgical instrumentation and in particular surgical tools intended to receive a cutting tool in a removable manner, for example a bistoury blade, in order to allow the surgeon to easily and rapidly change or choose the tool before or during a surgical operation so as to best carry out said surgical operation.

More particularly, the present invention concerns a surgical instrument intended to receive a cutting tool in a removable manner, for example a bistoury blade, including:

a receiving member, which in turn includes an elongate primary main body with a primary distal end, which is provided with a means for receiving the is cutting tool, said receiving means being designed to evolve between a locked configuration and an unlocked configuration,

a locking/unlocking device, which allows making the receiving means evolve from its locked configuration to its unlocked configuration and vice versa, said locking/unlocking device comprising a first element linked to the receiving member by a helical connection and a second element, comprising a secondary main body presenting an outer wall, linked to the receiving member by a sliding connection, so that a relative rotation of the first and second elements results in a relative translation of the receiving member and the second element thereby allowing controlling the locking/unlocking of the receiving means.

The present invention also concerns a method for manufacturing such a surgical instrument.

The present invention further concerns a surgical kit intended for the implementation of an operative technique, preferably percutaneous, in particular in the context of the treatment of a pathology of the foot of a patient, comprising such a surgical instrument.

In the field of surgical instruments intended to receive a cutting tool in a removable manner, for example a bistoury blade, there are known tools of the type [symbol] bistoury handle [symbol] composed of an elongate cylindrical handle, generally made of a metal and reusable, and a clamping mandrel capable of receiving, in a removable manner, bistoury blades, for a single use or not, and in different shapes.

The mandrel, with substantially cylindrical shape, includes at one of its ends a conical head provided with one or several axial slot(s), forming jaw elements, intended to receive the stem of the blade, that is to say its non-cutting end. At its other end, the mandrel is provided with an outer thread. This mandrel is slidingly mounted in a longitudinal axial hole formed in the handle and at the bottom of which a twist is provided.

Once the stem of a bistoury blade is inserted in a slot of the mandrel, the latter is brought to slide and then is screwed inside the handle, until that the support of the head of the mandrel is brought to cooperate with the edge of the axial hole, so as to cause the jaw elements of the mandrel to tighten around the stem of the blade and therefore secure the removable blade to the handle so as to form a bistoury.

While such known instruments generally give satisfaction in the context of the implementation of [symbol] open [symbol] surgical techniques, that is to say techniques involving the practice of a wide and open incision of the skin and the soft tissues, the bistoury blade remaining most often visible at the end of the handle, their use is substantially more delicate in the context of the implementation of a percutaneous operative technique, for example in the context of the treatment of bone pathologies of the foot by percutaneous surgery.

Indeed, the surgeon then loses sight of the blade when the latter is completely inserted in the body of the patient through a very thin incision practiced in the skin and the soft tissues. In particular, unless removing momentarily the end of the bistoury from the body of the patient, the surgeon has no longer access to any information regarding the orientation of the blade with respect to the blade-holder handle, which may turn out to be very problematic, the surgeon then risks cutting surrounding soft tissues involuntarily.

Consequently, the invention aims to remedy to this major drawback, and to propose a surgical instrument, intended to receive a cutting tool in a removable manner, which is more ergonomic and safer, and particularly well adapted to a use in the context of a percutaneous operative technique.

Another object of the invention aims to propose a new surgical instrument, intended to receive a cutting tool in a removable manner, which is particularly safe to use.

Another object of the invention aims to propose a new method for manufacturing a surgical instrument, which is both simple and inexpensive to implement.

Another object of the invention aims to propose a new surgical instrument, intended to receive a cutting tool in a removable manner, which is relatively inexpensive to manufacture.

Another object of the invention aims to propose a new surgical kit intended for the implementation of an operative technique, preferably percutaneous, which is complete and ergonomic.

Another object of the invention aims to propose a new surgical kit intended for the implementation of an operative technique, preferably percutaneous, which is safe and inexpensive.

The objects assigned to the invention are achieved by means of a surgical instrument intended to receive a cutting tool in a removable manner, for example a bistoury blade, including:

a receiving member, which in turn includes an elongate primary main body with a primary distal end, which is provided with a means for receiving the cutting tool, said receiving means being designed to evolve between a locked configuration and an unlocked configuration,

a locking/unlocking device, which allows making the receiving means evolve from its locked configuration to its unlocked configuration and vice versa, said locking/unlocking device comprising a first element linked to the receiving member by a helical connection, and a second element, comprising a secondary main body presenting an outer wall, linked to the receiving member by a sliding connection, so that a relative rotation of the first and second elements results in a relative translation of the receiving member and the second element thereby allowing controlling the locking/unlocking of the receiving means,

characterized in that said first element constitutes a gripping handle of said surgical instrument and in that the locking/unlocking device is provided with a marker which reflects the position of the cutting tool with respect to the second element, said marker being positioned axially on said outer wall.

The objects assigned to the invention are also achieved by means of a method for manufacturing such a surgical instrument, characterized in that said surgical instrument is integrally made by molding.

The objects assigned to the invention are also achieved by means of a surgical kit intended for the implementation of an operative technique, preferably percutaneous, in particular in the context of the treatment of a pathology of the foot of a patient, comprising such a surgical instrument, and being characterized in that it also comprises at least a retractor, a rugine and a rasp.

Other objects and advantages of the invention will appear in more details upon reading the description that follows, as well as with reference to the appended drawings, provided only for an explanatory and non-restrictive purpose, in which:

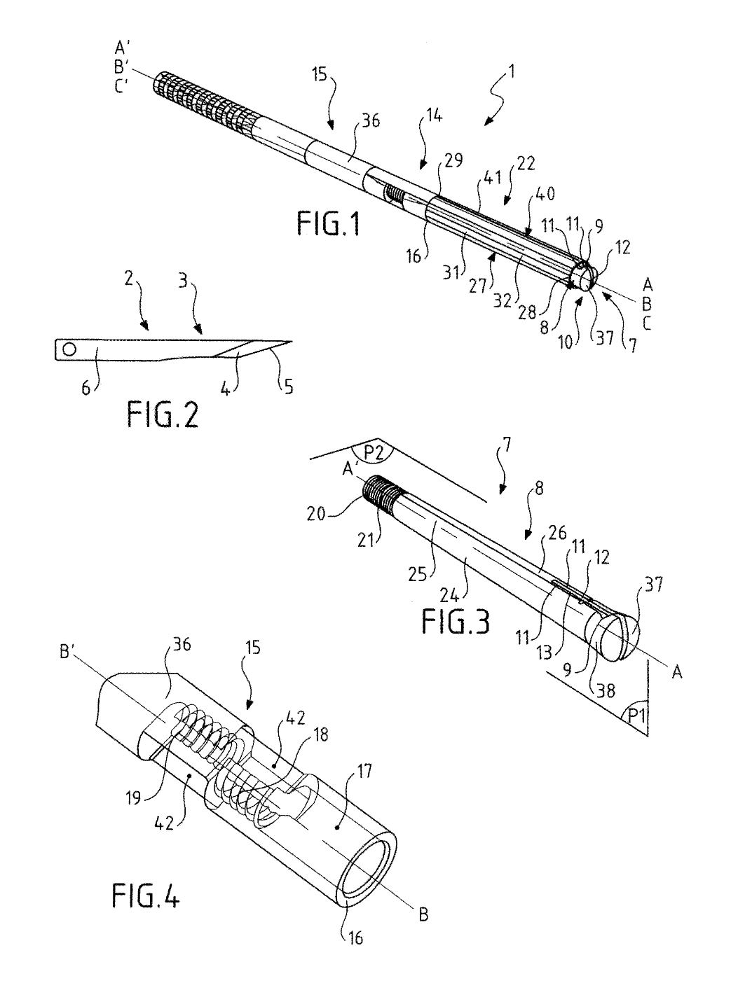

FIG. 1 illustrates, in a perspective view, a surgical instrument intended to receive a cutting tool in a removable manner in accordance with the invention;

FIG. 2 illustrates, in a profile view, an example of a cutting tool, in the form of a bistoury blade, capable of cooperating with the instrument of the invention;

FIG. 3 illustrates, in a perspective view, the receiving member of the surgical instrument of FIG. 1;

FIG. 4 illustrates, in phantom, the particular preferred embodiment of the twist, in the form of two half-twists, of the first element linked to the receiving member of the instrument of FIG. 1 by a helical connection;

FIG. 5 illustrates, in a perspective view, a preferred embodiment of the receiving member and the second element of the locking/unlocking device to which it is linked by a sliding connection;

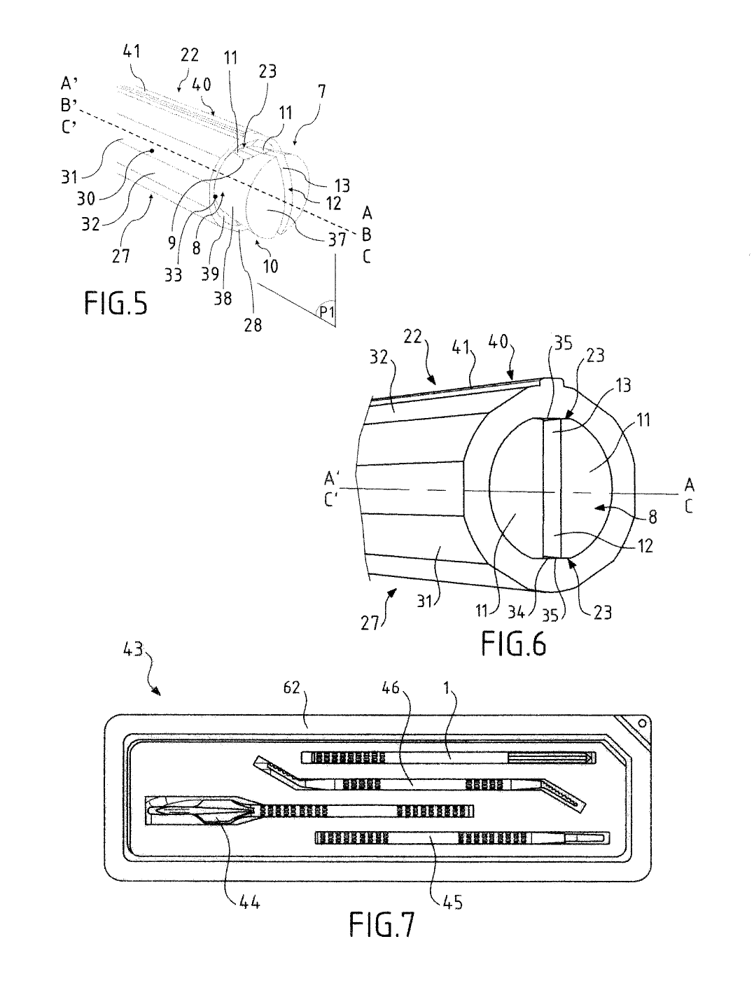

FIG. 6 illustrates, in a sectional view, the preferred embodiment of FIG. 5, so as to highlight the blocking device which allows realizing this sliding connection;

FIG. 7 illustrates, in a top view, a preferred embodiment of a surgical kit in accordance with the invention;

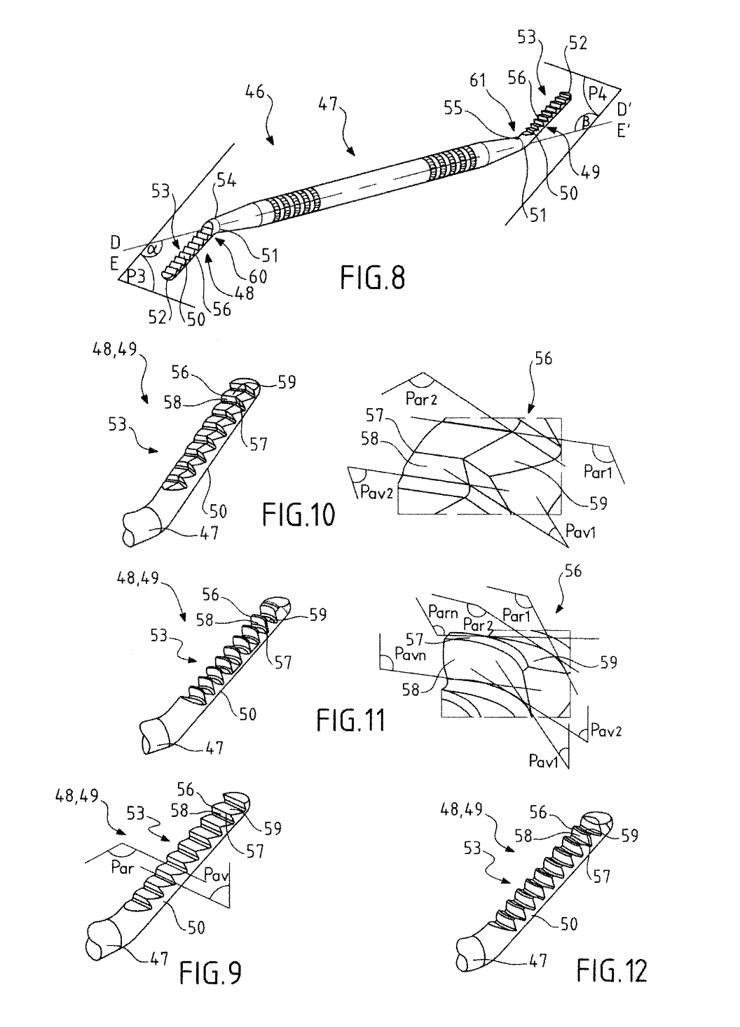

FIG. 8 illustrates, in a profile view, an embodiment of a rasp as preferably included in the surgical kit of FIG. 7;

FIGS. 9 to 12 illustrate different possible geometric configurations of the teeth of the rasp of FIG. 8.

The invention concerns a surgical instrument 1 intended to receive a cutting tool in a removable manner, for example a bistoury blade 2, one embodiment of which in accordance with the invention is illustrated in FIG. 1. In this instance, the surgical instrument 1 of the invention forms a blade holder handle, or [symbol] beaver [symbol] (by lexicalization of the trademark Beaver®) which is particularly adapted for a use by a surgeon during a percutaneous surgical operation on a patient, for example in the context of the treatment of a pathology of the foot, and in a more general manner, for treating pathologies which are simple or easily accessible (simple or slightly displaced fractures, tendon ruptures, various lesions though delimited, etc.) by percutaneous surgery. Of course, without departing from the scope of the invention, the surgical instrument 1 may be used in the context of a non-percutaneous operative technique, its implementation turning out to be particularly advantageous as long as there is a problem of visualization and accurate handling of the orientation of the cutting object attached thereto. Without restriction to an exclusive use on a human patient, the surgical instrument 1 may also be implemented in the context of an animal surgery, in a percutaneous way or not.

In the following, will be considered the example of a cutting object in the form of a bistoury blade 2, as illustrated in FIG. 2. Such a blade 2 includes a flat body 3 comprising a cutting head 4, provided with at least one cutting edge 5, prolonged by a rectangular stem 6. Of course, without departing from the scope of the invention, the considered cutting object may perfectly be in a form other than a bistoury blade 2, for example in the form of a sawtooth blade. Preferably, and as illustrated in FIG. 2, the cutting tool is therefore a substantially flat and elongate tool, and the instrument 1 is advantageously designed, as will be described hereinafter, to receive in a [symbol] straight [symbol] configuration, that is to say so that the cutting tool extends along the same average extension axis as the instrument, when assembled to the latter.

According to a major feature of the invention, the surgical instrument 1 includes a receiving member 7, which in turn includes an elongate primary main body 8, preferably extending along a first longitudinal axis A-A′, with a primary distal end 9, which is provided with a means 10 for receiving the cutting tool, for example a bistoury blade 2. Advantageously, said cutting tool is brought to interact with the receiving means 10 via one of its non-cutting ends, typically, in the case of a bistoury blade 2, via the stem 6 of said blade 2. Said receiving means 10 is designed to evolve between a locked configuration and an unlocked configuration, that is to say respectively between a configuration in which the cutting tool is firmly held in position by said receiving means 10 and a configuration in which, on the contrary, the cutting tool is free and can be detached from the receiving means 10.

Preferably, the receiving means 10 comprises at least two flexible receiving elements 11 defining an elongate axial slot 12 capable of receiving said cutting tool. This through slot 12, extending advantageously according to a first extension plane P1 in which said first longitudinal axis A-A′ is included, and this from said primary distal end 9 and over at least a portion of the length of the primary main body 8 of the receiving member 7. As illustrated in the figures, said receiving elements 11 then advantageously form jaw elements capable of evolving resiliently between a resilient biasing position in which the inner faces 13 of said slot 12 defined thereby naturally tend to remain parallel and away from each other, and a constrained position in which the receiving elements 11 are, on the contrary, bent so that said inner faces 13 tend to converge towards each other. Thus, when said receiving means 10 is in the locked configuration, the receiving elements 11 of the receiving means 10 adopt their constrained position and immobilize the portion of the cutting tool placed in said slot 12, by compression of the receiving elements between said inner faces 13.

According to a major feature of the invention, the surgical instrument 1 includes a locking/unlocking device 14, which allows making the receiving means 10 evolve from its locked configuration to its unlocked configuration and vice versa. Advantageously, this locking/unlocking device 14 allows securing the reception of the cutting tool by the receiving means 10 of the receiving member 7, in a reversible manner, by compelling the receiving elements 11 to adopt their constrained position in order to avoid that they unintendingly return to their resilient biasing position and release the cutting tool.

Said locking/unlocking device 14 comprises a first element 15, which preferably extends along a second longitudinal axis B-B′, linked to the receiving member 7 by a helical connection, in other words by a screw/nut type connection. In order to realize this helical connection, this first element 15 preferably includes a coupling end 16 provided with a twisted axial hole 17 the twist of which is constituted by two half-twists 18, 19. In other terms, and as illustrated in FIG. 4, the twist of the twisted axial hole 17 is not constituted by only one conventional full circular twist but, preferably, by the juxtaposition of two half-twists 18, 19, theses being positioned in an offset manner with respect to each other along the second longitudinal axis B-B′, opposite to each other at either side of the second longitudinal axis B-B′, and so that the end of the first half-twist 18 coincides with the beginning of the second half-twist 19. In turn, the receiving member 7 is preferably prolonged axially from a primary proximal end 20 by a threaded bar 21. The latter is advantageously designed so as to cooperate with the half-twists 18, 20 of said twisted axial hole 17, the first A-A′ and second B-B′ longitudinal axes then being preferably coincident. Thus, when the cutting tool forms a straight tool, which is neither recurved nor angled, the latter may be held straightly, that is to say so that its extension axis coincides with the first A-A′ and second B-B′ longitudinal axes.

According to the invention, and as will be progressively described and set out in detail hereinafter, the locking/unlocking device 14 comprises a second element 22 linked to the receiving member 7 by a sliding connection, so that a relative rotation of the first 15 and second 22 elements results in a relative translation of the receiving member 7 and the second element 22 thereby allowing controlling the locking/unlocking of the receiving means 10. Preferably, this sliding connection is realized from a sliding pivot connection relative to the axis A-A′ the rotation of which about said axis A-A′ is prevented by at least one blocking device 23.

According to the preferred embodiment illustrated in particular in FIG. 3, said primary main body 8 of the receiving member 7 is substantially cylindrical shaped, advantageously materializing a rod 24, and presents an outer surface 25, which advantageously links said primary distal end 9 to said primary proximal end 20. Said outer surface 25 is provided with at least one primary axial flat portion 26 extending:

longitudinally from said primary distal end 9 over at least a portion of the length of said primary main body 8 of the receiving member 7,

and according to a second extension plane P2 orthogonal to the first extension plane P1 of said slot 22.

This primary flat portion 26 forms a planar surface in the outer surface 25, characterized by a removal or absence of material, and which preferably extends according to a plane orthogonal to the first extension plane P1 of said slot 12. Preferably, it extends along the first longitudinal axis A-A′ towards said primary proximal end 20 of the primary main body 8.

Preferably, said second element 22 comprises a secondary main body 27, extending between a secondary distal end 28 and a secondary proximal end 29, provided with a cylindrical axial through hole 30, capable of receiving said primary main body 8 of the receiving member 7 tightly, that is to say almost without any clearance. Hence, said secondary main body 27 preferably extends along a third longitudinal axis C-C′ coincident with the first longitudinal axis A-A′ when said primary main body 8 is mounted in the cylindrical axial through hole 30 of said second element 22. According to the preferred embodiment illustrated in the figures, this secondary main body 27 is substantially cylindrical shaped and therefore forms a sleeve 31 inside which the primary main body 8 of the receiving member 7 can evolve. The outer wall 32 of the secondary main body 27, which preferably links said secondary distal end 28 and secondary proximal end 29, may advantageously be slightly fluted in order to guarantee, for the surgeon, a better manual gripping of the second element 22 of the locking/unlocking device 14 despite the use of medical gloves. Alternatively, without departing from the scope of the invention, it is perfectly conceivable that the secondary main body 27 of the second element 22 is for example in the general form of a right prism with a triangular, square or still hexagonal base, provided with a cylindrical axial through hole 30.

Preferably, said cylindrical axial hole 30 opens at said secondary distal end 28 through a distal orifice and at said secondary proximal end 29 through a proximal orifice (not represented). Thus, the primary main body 8 of the receiving member 7 is mounted in a removable manner with respect to the second element 22 and can evolve in the cylindrical axial hole 30 of the secondary main body 27 of the second element 22 between said distal 33 and proximal orifices. As illustrated in particular in FIG. 5, said cylindrical axial hole 30 defines an inner wall 34, which is advantageously provided with at least one secondary axial flat portion 35, preferably extending longitudinally from said secondary distal end 28 over at least a portion of the length of said secondary main body 27 of the second element 22, so that this secondary flat portion 35 forms, by cooperation with the primary flat portion 26, a blocking device 23 capable of preventing every rotation about the axis A-A′ of said receiving member 7 in said second element 22 of the locking/unlocking device 14.

Said primary main body 8 of the receiving member 7 being preferably mounted in the cylindrical axial hole 30 of said secondary main body 27 of the second element 22, so that the first A-A′ and third C-C′ longitudinal axes are coincident and that said at least one primary 26 and secondary 35 primary axial flat portions are positioned facing each other, as illustrated in particular in FIG. 5, the sliding pivot connection formed by said primary main body 8 of the receiving member 7 and said secondary main body 27 of the second element 22 is advantageously transformed into a sliding connection. Indeed, the translation along the first axis A-A′ of the primary main body 8 of the receiving member 7 in the cylindrical axial hole 30 of said secondary main body 27 of the second element 22 is enabled, whereas the rotation of said primary main body 8 about this first axis A-A′ is actually prevented by the cooperation of said primary 26 and secondary 35 flat portions.

In a particularly advantageous manner, and as is particularly visible in FIG. 5, said outer surface 25 of the primary main body 8 of the receiving member 7 is provided with two primary axial flat portions 26, and said inner wall 34 is provided with two secondary axial flat portions 35.

Said primary axial flat portions 26 and secondary axial flat portions 35 are respectively positioned radially opposite to each other, so that said secondary flat portions 35 form respectively, by cooperation with said primary flat portions 26, a blocking device 23 capable of preventing every rotation about the axis A-A′ of said receiving member 7 in said second element 22 of the locking/unlocking device 14.

This preferred variant with two sets of primary 26 and secondary 35 flat portions advantageously allows guaranteeing a perfect blocking of the rotation of the primary main body 8 of the receiving member 7 in the cylindrical axial hole 30 of said secondary main body 27 of the second element 22.

As described hereinabove, the second element 22 of the locking/unlocking device 27 is therefore linked to the receiving member 7 by a pure sliding connection. The latter is advantageously configured so that, when the threaded bar 21 of the primary main body 8 of the receiving member 7 is brought to cooperate with the twist of the twisted axial hole 17 of the first element 15 in order to link the latter to the receiving member 7 by a helical connection, a relative rotation of the first 15 and second 22 elements results in a pure translation along the first longitudinal axis A-A′ of the receiving member 7 in the second element 22.

Furthermore, when the receiving member 7 is mounted in the second element 22 of the locking/unlocking device 14, the angular position of said second element 22 with respect to the first longitudinal axis A-A′ permanently corresponds perfectly to the angular position of the receiving member 7 with respect to this first axis A-A′.

Alternatively, without departing from the scope of the invention, it is quite possible to consider that this pure sliding connection is, on the contrary, realized from a flat-plane connection to which is associated a second contact plane secant with the first one or, more simply, a lateral guide axis parallel to the plane.

Such a connection may then be realized by means of a primary main body 8 with a square or hexagonal section sliding in a second element 22 provided with an axial hole 30 respectively with a square or hexagonal section.

According to the preferred embodiment illustrated in FIG. 1, said first element 15 of the locking/unlocking device 14 advantageously constitutes a gripping handle 36 of said surgical instrument 1. Indeed, in this embodiment, said first element 15 extends longitudinally over a substantially larger length, for example more than twice longer, than the second element 22 of the locking/unlocking device 14, so as to form said gripping handle 36 in a practical manner.

Nonetheless, it is perfectly possible to consider an alternative embodiment, in which said first element 15 would, on the contrary, be substantially shorter than said second element 22. In this case, said second element 22 would then advantageously form the gripping handle 36, and the configuration of the receiving member 7 would be modified accordingly, still without impacting the technical features and functions of the surgical instrument 1 or altering its ergonomics.

Preferably, the receiving member 7 comprises a head 37, visible in FIGS. 1 and 3, larger than said primary main body 8. This head 37 prolongs the latter axially, that is to say preferably along the first longitudinal axis A-A′, from said primary distal end 9, and is formed by the flare of said first main body 8. In other terms, said head 37 is formed by the progressive enlargement of the section of the primary main body 8 in the direction of the secondary distal end 9.

In the preferred configuration described hereinbefore in which the primary main body 8 of the receiving member 7 is substantially cylindrical shaped, said head 37 presents a circular section with a diameter larger than the diameter of the average section of said primary main body 8. Moreover, the diameter of the circular section of said head 37 is advantageously larger than the diameter of the distal orifice 33 of the secondary distal end 9 of the secondary main body 27 of the second element 22.

As illustrated, said head 37 is advantageously integral with said receiving means 10, the slot 12 defined by the receiving elements 11 extending axially through said head 37. Still more preferably, the flare of said primary main body 8 forms a sloping bearing surface 38 of said head 37 intended to cooperate with an inner edge 39, preferably chamfered as illustrated in FIG. 5, of said distal orifice 33, so as to allow immobilizing the cutting tool in the receiving means 10, by compression between the receiving elements 11.

Thus, when the threaded bar 21 of the primary main body 8 of the receiving member 7 is brought to cooperate with the twist of the twisted axial hole 17 of the first element 15, a relative rotation of the first 15 and second 22 elements results in a translation, along the first longitudinal axis A-A′, of the receiving member 7 in the second element 22, until that, on the one hand, the secondary proximal end 29 of the secondary main body 27 of the second element 22 comes into contact with the coupling end 16 of the first element 15 and that, on the other hand, the bearing surface 38 of the head 37 is brought to cooperate with the inner edge 39 of the distal orifice 33 of the second element 22 in order to make the receiving elements 11 of the receiving means 10 evolve from their resilient biaising position to their constrained position in which they are bent so that their inner faces 13 tend to converge towards each other. In a corollary manner, a relative rotation of the first 15 and second 22 elements in the reverse direction results in a translation of the receiving member 7 in the second element 22, so as to disengage the contact between, on the one hand, the first 15 and second 22 elements and, on the other hand, the bearing surface 38 of the head 37 and the inner edge 39 of the distal orifice 33 of the second element 22 in order to make the receiving elements 11 of the receiving means 10 evolve from their constrained position to their resilient biaising position in which the inner faces 13 of said slot 12 defined thereby naturally tend to remain parallel and away from each other. Thus, this translation of the receiving member 7 in the second element 22 allows controlling respectively the locking and unlocking of the receiving means 10.

Preferably, the locking/unlocking device 14 is provided with a marker 40 of the position of the cutting tool with respect to the receiving member 7 and, still more advantageously, of the position of the cutting tool with respect to the second element 22. Thus, the cutting object is not only held firmly in the receiving means 10 of the receiving member 7 of the surgical instrument 1 in a determined position with respect to the second element 22 of the locking/unlocking device 14, but in addition, this determined position is reflected by said marker 40, so as to facilitate and ensafe the use of the surgical instrument 1 by the surgeon.

Said secondary main body 27 of the second element presenting, as introduced hereinbefore, an outer wall 32 which preferably links said secondary distal end 28 and secondary proximal end 29, said marker 40 is advantageously positioned axially, that is to say preferably along the third longitudinal axis C-C′, on said outer wall 32.

Preferably, the marker 40 is positioned so that, when the second element 22 is advantageously positioned between the cutting tool and the first element 15, it is closer to the cutting object than to first element which constitutes the gripping handle of the instrument 1. More preferably, said marker 40 extends longitudinally from said secondary distal end 28 over at least a portion of the length, and preferably over the entire length, of the secondary main body 27 of said second element 22. Thus, being advantageously positioned immediately proximate to the cutting tool in place in the receiving member 7, the marker 40 therefore allows an easy and rapid identification of the orientation of said cutting tool, since the rotation of the holding member 7 relative to the second element 22 is blocked when the cutting tool is locked. Accordingly, the surgical instrument 1 is particularly well adapted to be implemented in the context of a percutaneous operative technique, the marker 40 advantageously remaining perceptible by the surgeon even in the case of a deep surgical operation requiring the introduction of a portion of the second element 22 in the body of the patient through a thin incision practiced in his skin.

In a particularly advantageous manner, said marker 40 is positioned so as to face only one secondary flat portion 35. In other words, the marker 40 constitutes one single marking element 40 positioned on the outer wall 32 of the secondary main body 27 of the second element 22 of the locking/unlocking device 14, so as to faithfully track said secondary flat portion 35 provided in the inner wall 34 of said secondary main body 27 over all or part of its length. In the preferred case where said inner wall 34 is provided with two secondary axial flat portions 35, said marker 40 is then positioned so as to face only one of the two secondary axial flat portions 35. Accordingly, when said secondary flat portion 35, in front of which the marker 40 is positioned, cooperates with the, or with one of the, primary axial flat portion(s) 26 provided on the primary main body 8 of the receiving member 7, which primary flat portion 26 extends according to said second extension plane P2 orthogonal to the first extension plane P1 of said slot 12, the marker 40 then provides an information regarding the angular orientation of the first extension plane P1 of said slot 12 with respect to the first A-A′ and third C-C′ longitudinal axes.

In the example case where the cutting tool is a bistoury blade 2, the stem 6 of the latter being housed between the receiving elements 11 defining said slot 12 and the receiving means 10 being in its locked configuration, the marker 40 then allows identifying the angular orientation of the blade 2 with respect to the first A-A′ and third C-C′ longitudinal axes. Even when there is no direct visual contact with the blade 2, for example in the case of a percutaneous surgical operation, the surgeon then still knows the orientation of his blade 2. In order to benefit the best from the presence of this marker 40, the blade 2 is further positioned preferably between the receiving elements 11 of the receiving means 10, so that the cutting edge 5 of said blade 2 extends in a direction opposite to said marker 40 with respect to the first axis A-A′. In other terms, the cutting tool advantageously formed by the blade 2 extends along an axis coincident with the first axis A-A′, the marker 40 being positioned so as to face the back of said blade 2.

In a particularly advantageous manner, said marker 40 is a visual and tactile marker 40. In this manner, the marker 40 remains perfectly perceptible to the surgeon in every situation, in particular even in the case where the second element 22 of the locking/unlocking device 14 is fouled, for example by blood, and when the marker 40 is hardly visible to the eye. According to the preferred embodiment illustrated in FIGS. 1 and 5, and in order to ensure this double visual and tactile characteristic, said marker 40 is advantageously formed by a bead 41 of material protruding radially from said outer wall 32 of the secondary main body 27 of the second element 22. According to the preferred embodiment illustrated in the figures, this bead 41 is advantageously integral with the secondary main body 27 of the second element 22 and therefore constitutes one single piece with said secondary main body 27. Alternatively, it is nonetheless possible that this bead 41 constitutes a part distinct from said secondary main body 27 to which it would be attached for example by bonding or flush-fitting.

Preferably, the above-descried surgical instrument 1 is intended for a single use, that is to say that it is intended to be destroyed or recycled/valorized at the end of its use for a given patient and surgical operation. This allows not only a greater sanitary safety, but also a significant economic gain for the surgeon and the healthcare center in which he exerts, the costs related to the cleaning, sterilization and reconditioning of reusable surgical instruments being particularly high compared to the cost of the instrument itself. As such, said surgical instrument 1 is advantageously made of a polymer material, for example a polyacrylamide (PAA) based composite polymer material, the latter being optionally loaded, such as for example a polyacrylamide-based reinforced polymer material of the range [symbol] IXEF [symbol] ® commercialized by the company SOLVAY. Indeed, such a material has the advantage of being a biocompatible material having a good resistance to fatigue and a good tenacity. Optionally, it may be loaded with fibers, for example carbon or glass fibers, so as to confer to the surgical instrument 1 a great rigidity and an excellent mechanical bending strength. In addition, polyacrylamide-based materials are known to be particularly easy to implement, in particular by injection molding, even with a high fiber content. They present a small shrinkage at molding and therefore allow the accurate and repeatable realization of parts with small dimensions and thicknesses and complex shapes.

The invention further concerns, as such, a method for manufacturing such a surgical instrument 1 as described before. According to this method, said surgical instrument 1 is integrally made by molding. In other terms, each elements included by said surgical instrument 1, namely in particular the receiving member 7 and the first 15 and second 22 elements of the locking/unlocking device 14, is obtained by molding, for example by injection molding, in one single operation or through several distinct operations, and without any other finishing or recovery operation such as machining or others. Advantageously, the material intended to be molded during the manufacturing method is a polymer material, preferably a polyacrylamide (PAA) based composite polymer material as mentioned hereinbefore. Preferably, said manufacturing method is implemented exclusively by means of draft-molding tools. This means that said method preferably does not implement any molding tool presenting negative drafts, or undercuts. In particular, said method does not implement, in particular for the realization of the twisted axial hole 17 of the first element 15, any threaded core, which is expensive and complex to extract by rotation when demolding.

Preferably, the manufacture of the surgical instrument 1 proceeds in the following manner:

the material intended to form the receiving member 7 is poured or cast or injected in a first mold, formed for example by two mold-halves closing according to a first parting plane orthogonal to the first extension plane P1 of the slot 12,

the material intended to form the first element 15 of the locking/unlocking device 14 is poured or cast or injected in a second mold, preferably with drawers, formed for example by two mold-halves closing according to a second parting plane containing the first longitudinal axis B-B′, this second mold comprising:

a first cylindrical draft drawer, positioned along the second longitudinal axis B-B′ coincident with said second parting plane, allowing forming an axial hole 17 in the coupling end 16 of said first element 15,

a second and a third drawers with a rectangular section and carrying at one of their ends a half-twist cavity, positioned in a plane orthogonal to said second parting plane.

the material intended to form the second element 22 of the locking/unlocking device 14 is poured or cast or injected in a third mold, formed for example by two mold-halves closing according to a third parting plane orthogonal to the longitudinal axis C-C′ along which said second element 22 extends.

The implementation of the method of the invention in particular to realize the first element 15 of the locking/unlocking device 14 is reflected, in a particularly obvious manner, by the creation of two through apertures 42 (visible in FIG. 4), substantially rectangular and offset with respect to each other along the second longitudinal axis B-B′, and formed through the surface of the first element 15 according to extension planes parallel to said second parting plane. Each of these apertures 42 respectively offers a view on one of the half-twists 18, 19 forming the twist of the twisted axial hole 17 of the first element 15 of the locking/unlocking device 14.

The invention also concerns, as such, a surgical kit 43 intended for the implementation of an operative technique, preferably percutaneous, in particular in the context of the treatment of a pathology of the foot of a patient, comprising a surgical instrument 1 in accordance with the invention, and as described hereinbefore, and also comprising at least a retractor 44, a rugine 45 and a rasp 46.

The retractor 44 is a surgical tool, in the general form of a lever, intended to allow modifying the relative positioning of bone bodies of a patient. Such a retractor 44 conventionally consisting of a rod terminated by a thinner and curved head so as to form a [symbol] Z [symbol] shape with the rod. Nonetheless, the retractor 44 comprised in the surgical kit 43 of the invention preferably includes a gripping handle linked to a recurved working head provided with an axial groove in particular capable of receiving and guiding an osteosynthesis wire, for example Kirschner wires. Furthermore, this working head may advantageously be provided with incurved longitudinal flanges, so as to offer a wide and stable support to the retractor 44 when the latter is maneuvered like a lever in order to modify the positioning of said bone bodies.

The rugine 45 is a surgical tool intended to scrape off a bone surface and, in particular, to separate from the bones the surrounding soft tissues such as muscles or tendons, in order to enable working in direct contact with said bone surface. Such a rugine 45 is formed by a small plate supported by a handle. Said plate is provided with sharp ridges and its free end, which forms a ridge perpendicular to the main axis of the handle, is beveled so as to form a leading edge specifically aggressive in order to ensure the detachment of the periosteum and the soft tissues. Preferably, said plate and said handle form one single piece. Nonetheless, said rugine 45 may possibly be formed by distinct plate and handle, said plate being inserted in the handle in a permanent or still in a removable manner.

The rasp 46 (or [symbol] excavator [symbol] ), one embodiment of which is illustrated in FIG. 8, is a surgical tool which allows extracting from the body of the patient biological debris or sequestrum, constituted in particular by bone or tissular fragments mixed with blood, in particular those generated during an osteotomy operation, for example of the first metatarsus, or during a reduction of the volume of the metatarsal head. Preferably, such a rasp 46 includes:

on the one hand, a rasp handle 47 extending along a fourth longitudinal axis D-D′, and

on the other hand, at least one rasp head 48, 49, including an elongate body 50 extending along a fifth longitudinal axis E-E′ between a first end 51 linked to the rasp handle 47 and a second free end 52, said body 50 presenting an active face 53 linking said first 51 and second 52 ends.

Advantageously, said at least one rasp head 48, 49 extends in the extension of the rasp handle 47, preferably from one of the ends 54, 55 of the latter, said fourth D-D′ and fifth E-E′ axes being preferably coincident.

Said active face 53 of the rasp head 48, 49 is provided with a row of parallel teeth 56, aligned along the fifth longitudinal axis E-E′, and presenting sharp ridges 57 capable of effectively rasping the bone bodies and the surrounding soft tissues in order to extract the sequestrum. Conventionally, these teeth 56 are arranged in a [symbol] straight [symbol] configuration, illustrated in FIG. 9, that is to say that they extend linearly in a direction orthogonal to the fifth longitudinal axis E-E′, their front 58 and rear 59 faces being respectively inscribed in one single plane Pay, Par.

Preferably, the front 58 and rear 59 faces of said teeth 56 are respectively inscribed in at least two distinct secant planes Pav1, Pav2, Par1, Par2, so that said teeth 56 may advantageously present an alternative geometric configuration at least among the following ones:

a [symbol] chevron [symbol] type configuration: according to this first variant, illustrated in FIG. 10, the front 58 and rear 59 faces of said teeth 56 are inscribed respectively in two distinct secant planes Pav1, Pav2, Par1, Par2, so that said teeth 56 present a [symbol] V [symbol] -shaped concave profile, the opening of the [symbol] V [symbol] shape preferably opening in the direction of the rasp handle 47;

a first [symbol] concave [symbol] configuration: according to this second variant, illustrated in FIG. 11, the front 58 and rear 59 faces of said teeth 56 are preferably inscribed respectively in a multitude of distinct secant planes Pav1, Pav2, . . . , Pavn, Par1, Par2, . . . , Parn, so that said teeth 56 present a curvilinear concave profile, the concavity of the teeth 56 opening preferably in the direction of the rasp handle 47;

a second [symbol] concave [symbol] configuration: according to this third variant, illustrated in FIG. 12, the front 58 and rear 59 faces of said teeth 56 are preferably inscribed in a multitude of distinct secant planes Pav1, Pav2, . . . ,Pavn, Par1, Par2, . . . , Parn, so that said teeth 56 present a curvilinear concave profile, the concavity of the teeth 56 opening preferably in the direction opposite to that of the rasp handle 47.

Indeed, it has been observed that these [symbol] chevron [symbol] type and concave configurations, in comparison with the conventional [symbol] straight [symbol] configuration, advantageously allow blocking more bone sequestra in the concavity of each tooth and thus extracting them from the open wound more effectively and rapidly, which contributes to the reduction of the operative time. Of course, the teeth 56 of said rasp head 48, 49, may present advantageous variants of the geometric configuration others than those described hereinabove, for example a configuration according to a [symbol] W [symbol] -shaped profile.

Preferably, the body 50 of said rasp head 48, 49 is firmly linked at its first end 51 to the rasp handle 47, said rasp head 48, 49 and said rasp handle 47 being made secured to each other. Thus, the rasp head 48, 49 may constitute a part independent from the rasp handle 47, possibly interchangeable, affixed and firmly held to the latter for example by bonding, screwing, clipping or still by plugging. Nonetheless, preferably, said rasp handle 47 and said rasp head 48, 49 are integral with each other, that is to say that they advantageously constitute one single piece completely made of a given material, preferably a polymer material, a polyacrylamide (PAA) based composite polymer material, the latter being optionally loaded.

Preferably, and as illustrated in FIG. 8, said rasp 46 advantageously includes two rasp heads 48, 49, each linked to one of the ends 54, 55 of the rasp 47 handle.

The bodies 50 of the rasp heads 48, 49 extend respectively substantially along a first P3 and a second P4 extension plane secant to the fourth longitudinal axis D-D′, the bodies 50 of said rasp heads 48, 49 and the rasp handle 47 being respectively linked by a first 60 and a second 61 inflection area.

In a preferred variant illustrated in FIG. 8, said rasp 46 includes two rasp heads 48, 49. These extend respectively in a third P3 and in a fourth P4 extension planes, said planes P3, P4 being parallel and secant to the fourth longitudinal axis D-D′ so as to form with the latter an angle of elevation α, β preferably comprised between 135° and 165°. Furthermore, in this preferred variant, said rasp heads 48, 49 advantageously extend in opposite directions, thereby conferring to said rasp a [symbol] Z [symbol] -shape, so that the surgeon is not bothered by the presence of the other rasp head 48, 49, when he manually uses either one of the rasp heads 48, 49. Nonetheless, other variants may be perfectly considered, the third P3 and fourth P4 extension planes may be not parallel and the angles of elevation α, β respectively formed by these planes with the fourth longitudinal axis D-D′ may be identical or different, while advantageously being comprised between 135° and 165°.

Lastly, when said rasp 46 is in a variant in which it includes two rasp heads 48, 49, the teeth 56 of these may present geometric configurations, as described before, identical or still different, so as to offer to the surgeon a rasp 46 which is perfectly adapted to his needs.

It is understood that the above-described rasp 46, considered independently from said surgical kit 43, may constitute, as such, an invention on its own, distinct from the present invention.

Without departing from the scope of the invention, the surgical kit 43 may also include surgical tools others than those described hereinabove, for example one or several bistoury blade(s) 2, one or several osteosynthesis wire(s), for example Kirschner wires, or still one or several percutaneous burr(s) allowing realizing bone cuts.

As illustrated in FIG. 7, said kit 43 is advantageously constituted by a package 62, made for example of a polypropylene-type transparent plastic, in which are disposed said surgical instrument 1, retractor 44, rugine 45 and rasp 46, these being preferably cushioned in said package 62 by means of a cushioning foam.

Preferably, said surgical kit 43 is intended for a single use, that is to say that said surgical instrument 1, retractor 44, rugine 45 and rasp 46 composing it are intended to be destroyed or recycled/valorized at the end of their use for a given patient and surgical operation. However, without departing from the scope of the invention, said kit 43 may on the contrary be reusable, that is to say that said surgical instrument 1, retractor 44, rugine 45 and rasp 46 composing it may be supplied again, after a first use, to the surgeon after having undergone a retreatment and an adequate reconditioning. As such, said surgical instrument 1, retractor 44, rugine 45 and rasp 46 are advantageously all made of a polymer material, for example a polyacrylamide (PAA) based composite polymer material, the latter being optionally loaded. Still more preferably, said surgical instrument 1, retractor 44, rugine 45 and rasp 46 are advantageously conditioned in a sterile manner in the package 62, so as to be immediately usable by the surgeon without a preliminary cleaning and sterilization step.

In fine, when equipped with a cutting tool, for example a bistoury blade 2, the surgical instrument 1 of the invention, particularly ergonomic and safe to use, allows the surgeon to carry out a percutaneous operative technique with a minimum of risk of involuntarily damaging the surrounding soft tissues. Made by a simple manufacturing method, the surgical instrument 1 is relatively inexpensive to produce. Furthermore, it is advantageously integrated in the surgical kit 43 of the invention, so as to offer to the surgeon a complete, ergonomic, safe and inexpensive set of tools intended for the implementation of an operative technique, preferably percutaneous, for treating a bone pathology, in particular of the foot.

1. A surgical instrument (1) intended to receive a cutting tool in a removable manner, for example a bistoury blade (2), including:
a receiving member (7), which in turn includes an elongate primary main body (8) with a primary distal end (9), which is provided with a means (10) for receiving the cutting tool, said receiving means (10) being designed to evolve between a locked configuration and an unlocked configuration,
a locking/unlocking device (14), which allows making the receiving means (10) evolve from its locked configuration to its unlocked configuration and vice versa, said locking/unlocking device (14) comprising a first element (15) linked to the receiving member (7) by a helical connection, and a second element (22), comprising a secondary main body (27) presenting an outer wall (32), linked to the receiving member (7) by a sliding connection, so that a relative rotation of the first (15) and second (22) elements results in a relative translation of the receiving member (7) and the second element (22) thereby allowing controlling the locking/unlocking of the receiving means (10),
characterized in that said first element (15) constitutes a gripping handle (36) of said surgical instrument (1) and in that the locking/unlocking device (14) is provided with a marker (40) which reflects the position of the cutting tool with respect to the second element (22), said marker (40) being positioned axially on said outer wall (32).

2. The surgical instrument (1) according to claims claim 1, characterized in that the receiving means (10) comprises at least two flexible receiving elements (11) defining an elongate axial slot (12) capable of receiving said cutting tool.

3. The surgical instrument (1) according to claim 2, characterized in that said primary main body (8) of the receiving member (7) is substantially cylindrical shaped and presents an outer surface (25), which outer surface (25) is provided with at least one primary axial flat portion (26) extending:
longitudinally from said primary distal end (9) over at least a portion of the length of said primary main body (8) of the receiving member (7),
and according to a second extension plane (P2) orthogonal to the first extension plane (P1) of said slot (22).

4. The surgical instrument (1) according to claim 3, wherein the secondary main body (27) of said second element (22), extending between a secondary distal end (28) and a secondary proximal end (29), said secondary main body (27) is provided with a cylindrical axial through hole (30) capable of tightly receiving said primary main body (8) of the receiving member (7), said cylindrical axial hole (30) opening from said secondary distal end (28) through a distal orifice (33) and from said secondary proximal end (29) through a proximal orifice, and wherein said cylindrical axial hole (30) defines an inner wall (34), which is provided with at least one secondary axial flat portion (30), so that this secondary flat portion (35) forms, by cooperation with the primary flat portion (26), a blocking device (23) capable of preventing every rotation about the axis A-A′ of said receiving member (7) in said second element (22) of the locking/unlocking device (14).

5. (canceled)

6. The surgical instrument (1) according to claim 2, characterized in that:
said outer surface (25) of the primary main body (8) of the receiving member (7) is provided with two primary axial flat portions (26), and
said inner wall (34) is provided with two secondary axial flat portions (35),
said primary axial flat portions (26) and secondary axial flat portions (35) being respectively positioned radially opposite to each other, so that said secondary flat portions (35) form respectively, by cooperation with said primary flat portions (26), a blocking device (23) capable of preventing every rotation about the axis A-A′ of said receiving member (7) in said second element (22) of the locking/unlocking device (14).

7. The surgical instrument (1) according to claim 1, characterized in that said secondary main body (27) of the second element (22) extends between a secondary distal end (28) and a secondary proximal end (29), said marker (40) extending longitudinally from said secondary distal end (28) over at least a portion of the length, and preferably over the entire length, of the secondary main body (27) of said second element (22).

8. The surgical instrument (1) according to claim 1, characterized in that said marker (40) is a visual and tactile marker (40).

9. The surgical instrument (1) according to claim 8, characterized in that said marker (40) is formed by a bead (41) of material protruding radially from said outer wall (32) of the secondary main body (27) of the second element (22).

10. The surgical instrument (1) according to claim 4, characterized in that said marker (40) is positioned so as to face only one secondary flat portion (35).

11. The surgical instrument (1) according to claim 1, characterized in that the receiving member (7) comprises a head (37) larger than said primary main body (8), prolonging the latter axially from said primary distal end (9), and formed by the flare of said primary main body (8).

12. The surgical instrument (1) according to claim 10, characterized in that the flare of said first main body (8) forms a sloping bearing surface (38) of said head (37) intended to cooperate with an inner edge (39) of said distal orifice (33), so as to allow immobilizing the cutting tool in the receiving means (10), by compression between the receiving elements (11).

13. The surgical instrument (1) according to claim 1, characterized in that the receiving member (7) is prolonged axially from a primary proximal end (20) by a threaded bar (21).

14. The surgical instrument (1) according to claim 13, characterized in that said first element (15) includes a coupling end (16) provided with a twisted axial hole (17) the twist of which is constituted by two half-twists (18, 19) positioned in an offset manner with respect to each other.

15. The surgical instrument (1) according to claim 1, characterized in that it is intended for a single use.

16. The surgical instrument (1) according to claim 1, characterized in that it is made of a polymer material, for example a polyether-ether-ketone PEEK, the latter being optionally loaded.

17. A method for manufacturing such a surgical instrument (1) according to claim 1, characterized in that said surgical instrument (1) is integrally made by molding.

18. The method for manufacturing a surgical instrument (1) according to claim 19, characterized in that it is implemented exclusively by means of draft-molding tools.

19. A surgical kit (43) intended for the implementation of an operative technique, preferably percutaneous, in particular in the context of the treatment of a pathology of the foot of a patient, comprising a surgical instrument (1) according to claim 1, and being characterized in that it also comprises at least a retractor (44), a rugine (45) and a rasp (46).

20. The surgical kit (43) according to claim 19, characterized in that it is intended for a single use.

21. The surgical kit (43) according to claim 19, characterized in that said surgical instrument (1), retractor (44), rugine (45) and rasp (46) are all made by molding of a polymer material, for example a polyether-ether-ketone PEEK, the latter being optionally loaded with carbon.
